(19) United States
(12) Patent Application Publication
(10) Pub. No.: US 2006/0292158 A1
(43) Pub. Date: Dec. 28, 2006

(54) Method for the treatment of cancer using a novel pharmaceutical composition containing at least one dolastatin 10 derivative

(76) Inventors: Yuko Aoki, Chigasaki-shi (JP); Masanori Miwa, Kamakura-shi (JP); Fumie Sawamura, Yokohama-shi (JP); Hiromi Tanimura, Fujisawa-shi (JP); Toshikazu Yamazaki, Kamakura-shi (JP)

Correspondence Address: HOFFMANN-LA ROCHE INC.;PATENT LAW DEPARTMENT, 340 KINGSLAND STREET, NUTLEY, NJ, 07110, US

(21) Appl. No.: 11/297,716

(22) Filed: Dec. 8, 2005

(30) Foreign Application Priority Data
Dec. 13, 2004 (EP) 04106522.8
Feb. 8, 2005 (EP) 05100866.2

(51) Int. Cl. A61K 38/06; A61K 39/395; A61K 31/675; A61K 33/24; A61K 31/7072

(52) U.S. Cl. 424/155.1; 514/18; 514/94; 514/283; 424/649

(57) ABSTRACT

The present invention is directed to a method for the treatment of cancer via the administration of a pharmaceutical composition, comprising at least one compound of formula (I) 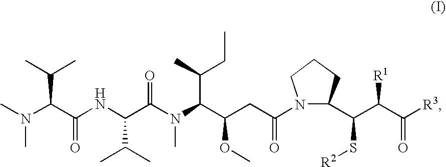 or a pharmaceutically acceptable salt of compound (I), in combination with capecitabine, trastuzumab, pertuzumab, cisplatin or irinotecan, or a pharmaceutically acceptable salt thereof, for simultaneous, sequential or separated administration in the treatment of cancer.

### PRIORITY TO RELATED APPLICATIONS

This application claims the benefit of European Application No. 04106514.5, filed Dec. 13, 2004, which is hereby incorporated by reference in its entirety.

### BACKGROUND OF THE INVENTION

Dolastatin 10 is known to be a potent antimitotic peptide, isolated from the marine mollusk Dolabella auricularia, which inhibits tubulin polymerization and is a different chemical class from taxanes and vincas (Curr. Pharm. Des. 1999, 5: 139-162). Preclinical studies of Dolastatin 10 have demonstrated activities against a variety of murine and human tumor cell lines in culture and in xenograft tumor models in animals. Dolastatin 10 and two synthetic dolastatin derivatives, Cemadotin and TZT-1027 are described in Drugs of the future 1999, 24(4): 404-409.

Subsequently it had been found that the Dolastatin 10 derivatives of formula (I), having various thio-groups at the dolaproine part show significantly improved anti-tumor activity and therapeutic index in human cancer xenograft models (WO 03/008378).

Chemotherapy and more particularly combined chemotherapy is one of the means well accepted to fight cancers. Thus the combination of different antitumor agents may be a way to increase the antitumoral efficacy when a more than additive effect is revealed and/or less toxicity is observed.

### SUMMARY OF THE INVENTION

It has now been found that a combination of at least one Dolastatin 10 derivative, in particular at least one of formula (I) as defined below, shows more than additive effects when administered together with the known anti cancer drugs Xeloda™, Herceptin™, Omnitarg™, Camptosar™ (Topotecin™ in Japan) or PlatinoL™ (Randa™ in Japan).

Therefore, the present invention is directed to a method for the treatment of cancer via administration of a pharmaceutical composition, comprising at least one compound of formula (I) or a pharmaceutically acceptable salt thereof 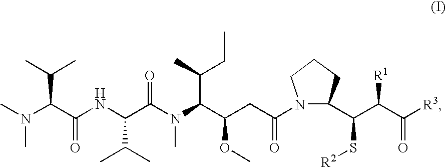
in combination with capecitabine; trastuzumab; pertuzumab; irinotecan or a pharmaceutically acceptable salt thereof; or cisplatin for simultaneous, sequential or separated administration in the treatment of cancer;
wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

In a preferred embodiment, the present invention is directed to a method for the treatment of cancer via administration of a pharmaceutical composition, comprising at least one compound of formula (I) or a pharmaceutically acceptable salt thereof 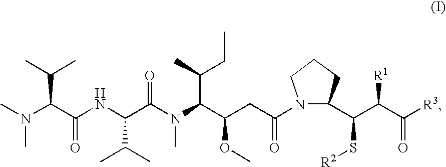
in combination with capecitabine, trastuzumab or pertuzumab for simultaneous, sequential or separate administration in the treatment of cancer; wherein
R¹ and R² are methyl; ethyl; propyl; isopropyl or butyl; R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

### DESCRIPTION OF THE DRAWINGS

FIG. 1a: Mean Tumor Volume (T.V.; mm³) versus days after the initial treatment of KPL-4 tumor cells with the compound of formula I-A (3.0 mg/kg) alone and in combination with trastuzumab (Herceptin™; Her; 20 mg/kg). The data for the treatment with trastuzumab alone (20mg/kg) as well as for the vehicle are also given.

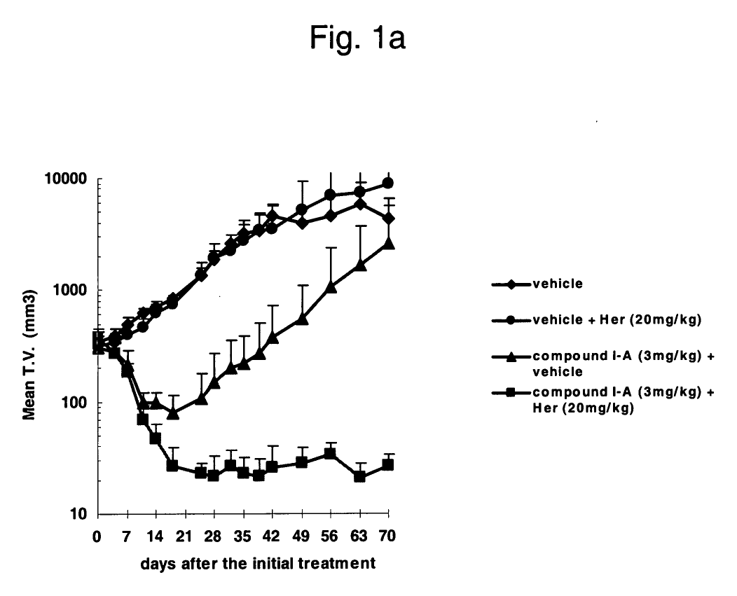

FIG. 1b: Mean Tumor Volume (T.V.; mm³) versus days after the initial treatment of KPL-4 tumor cells with the compound of formula I-A (4.0 mg/kg) alone and in combination with trastuzumab (Herceptin; Her; 20 mg/kg). The data for the treatment with trastuzumab alone (20mg/kg) as well as for the vehicle are also given.

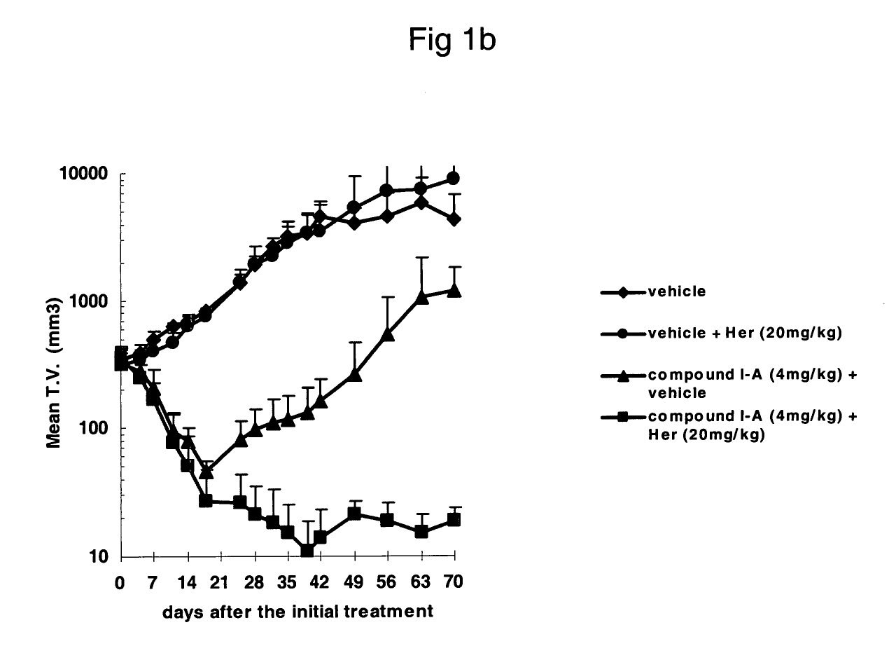

FIG. 1c: Mean Tumor Volume (mm³) versus days after the initial treatment of KPL-4 tumor cells with the compound of formula I-A (6.0 mg/kg) alone and in combination with trastuzumab (Herceptin™; Her; 20 mg/kg). The data for the treatment with trastuzumab alone (20mg/kg) as well as for the vehicle are also given.

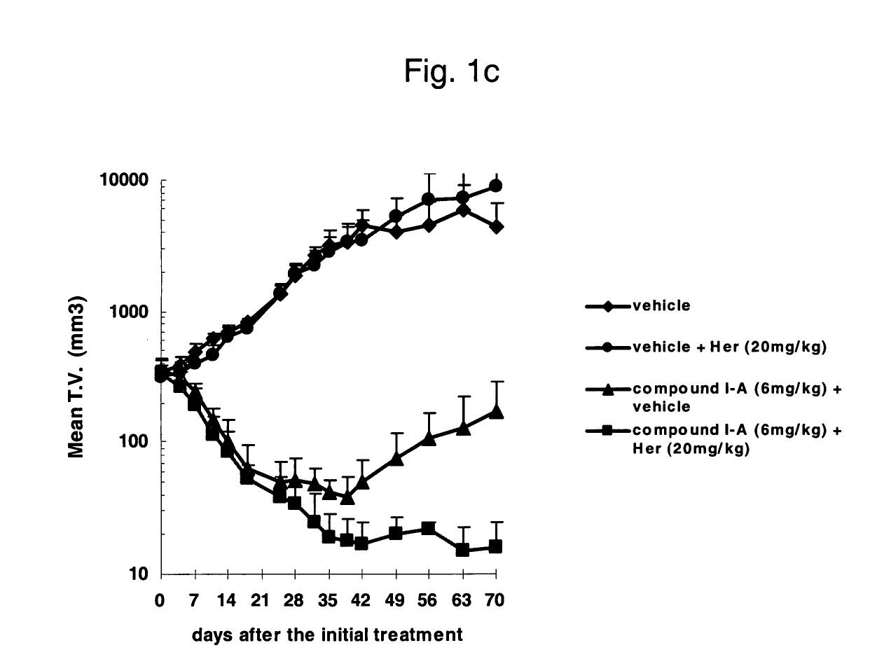

FIG. 2a: Mean Tumor Volume (T.V., mm³) versus days after the initial treatment of KPL-4 tumor cells with the compound of formula I-A (3.0 mg/kg) alone and in combination with pertuzumab (Omnitarg™; 2C4; 30 mg/kg). The data for the treatment with pertuzumab alone (30 mg/kg) as well as for the vehicle are also given.

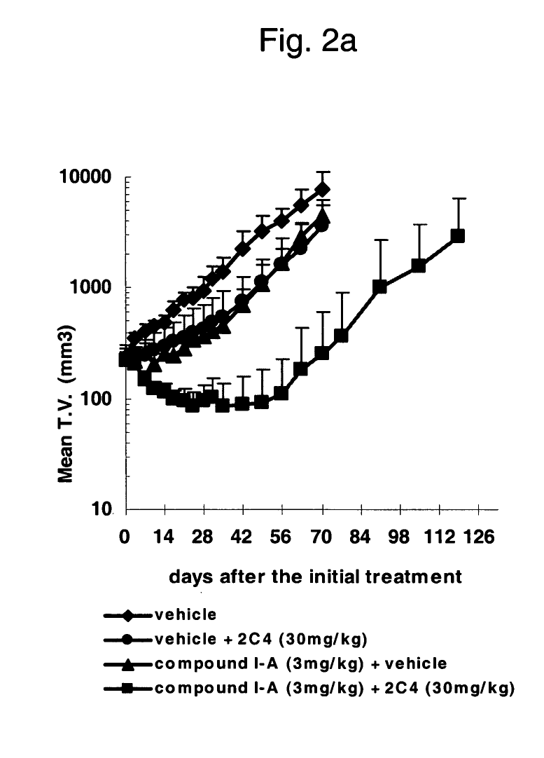

FIG. 2b: Mean Tumor Volume (T.V., mm³) versus days after the initial treatment of KPL-4 tumor cells with the compound of formula I-A (4.0 mg/kg) alone and in combination with pertuzumab (Omnitarg™; 2C4; 30 mg/kg). The data for the treatment with pertuzumab alone (30mg/kg) as well as for the vehicle are also given.

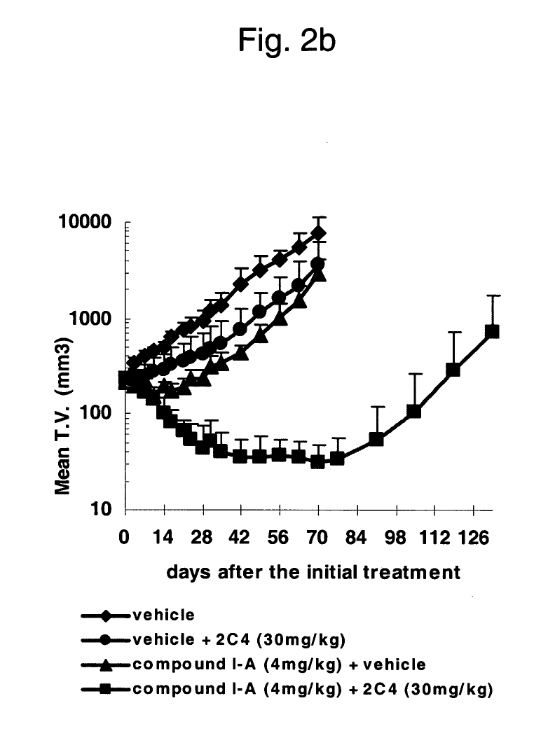

FIG. 2c: Mean Tumor Volume (T.V., mm³) versus days after the initial treatment of KPL-4 tumor cells with the compound of formula I-A (6.0 mg/kg) alone and in combination with pertuzumab (Omnitarg™; 2C4; 30 mg/kg). The data for the treatment with pertuzumab alone (30mg/kg) as well as for the vehicle are also given.

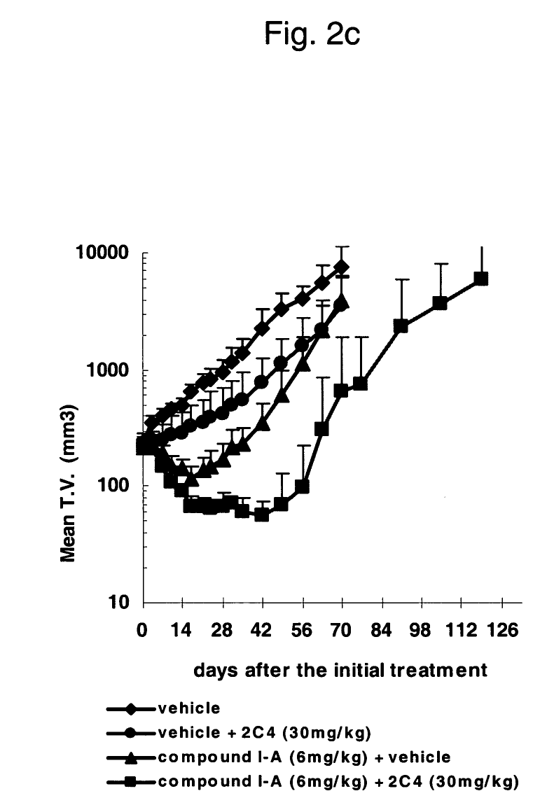

FIG. 3a: Tumor Volume (mm³) of NCI-H460 tumor cells versus days after inoculation with the compound of formula I-A (2mg/kg) alone and in combination with cisplatin (Randal™; CDDP; 6.7mg/kg). The data for the treatment with cisplatin alone (Randa™; CDDP; 6.7mg/kg) as well as for the vehicle are also given.

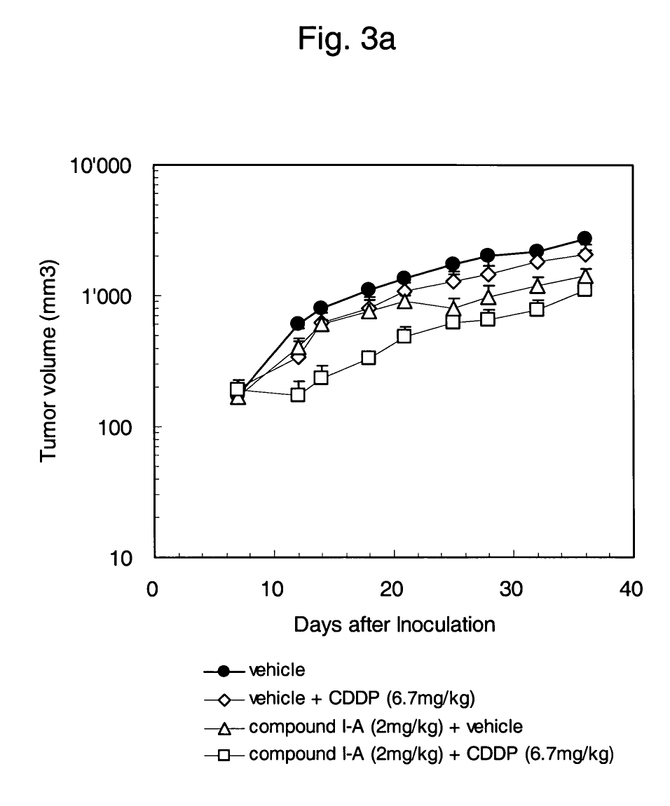

FIG. 3b: Tumor Volume (mm³) of NCI-H460 tumor cells versus days after inoculation with the compound of formula I-A (3mg/kg) alone and in combination with cisplatin (Randal™; CDDP; 5mg/kg). The data for the treatment with cisplatin alone (Randal™; CDDP; 5mg/kg) as well as for the vehicle are also given.

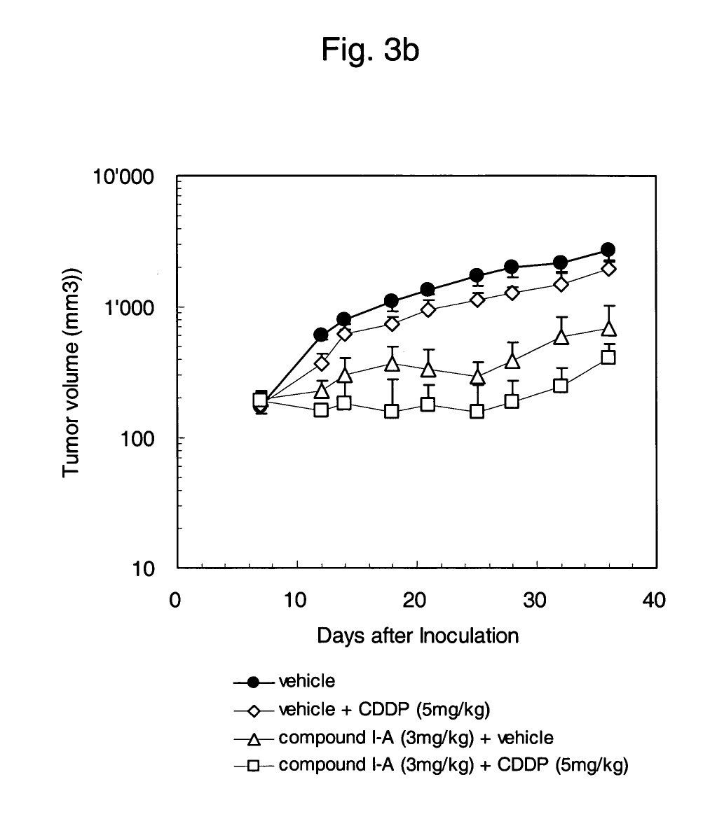

FIG. 4: Tumor Volume (mm³) of Calu-6 tumor cells versus days after inoculation with the compound of formula I-A (1.5 mg/kg) alone and in combination with irinotecan hydrochloride (Topotecin™; CPT-11; 80 mg/kg). The data for the treatment with irinotecan hydrochloride alone (Topotecin™; CPT-11; 80 mg/kg) as well as for the vehicle are also given.

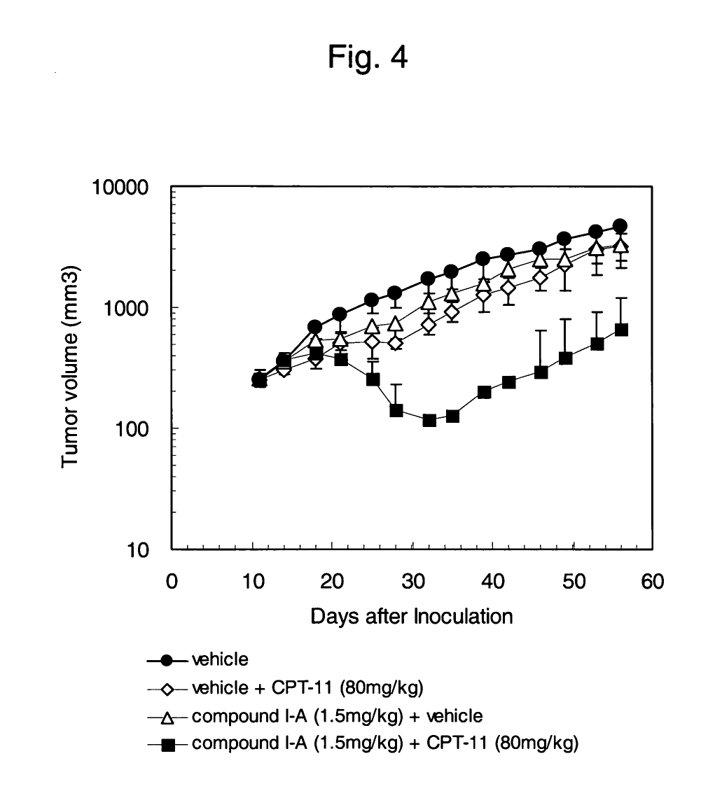

FIG. 5a: Tumor Volume (mm³) of HT-29 tumor cells versus days after inoculation with the compound of formula I-A (3 mg/kg) alone and in combination with capecitabine (Xeloda™; 539 mg/kg). The data for the treatment with capecitabine alone (Xeloda™; 539 mg/kg) as well as for the vehicle are also given.

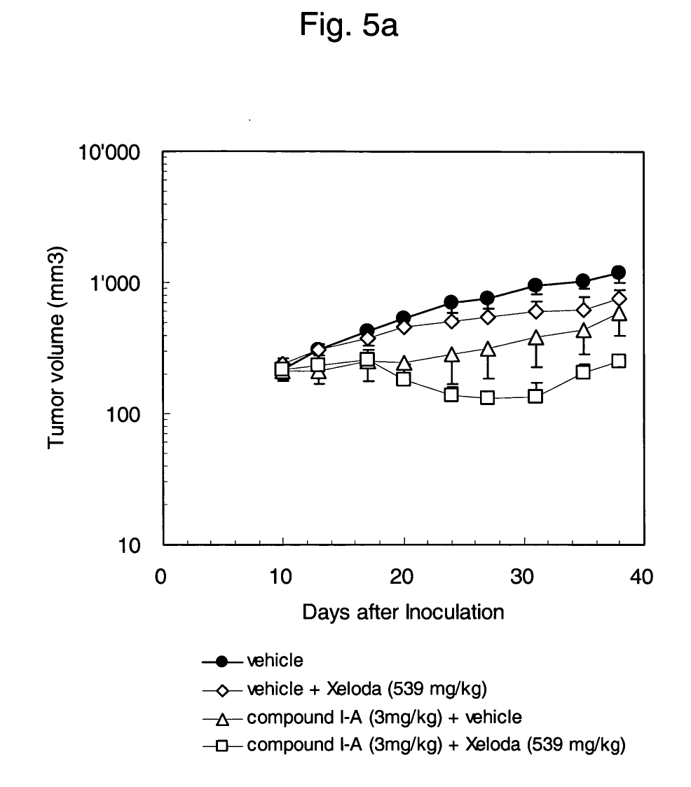

FIG. 5b: Tumor Volume (mm³) of HT-29 tumor cells versus days after inoculation with the compound of formula I-A (4 mg/kg) alone and in combination with capecitabine (Xeloda™; 359 mg/kg). The data for the treatment with capecitabine alone (Xeloda™; 359 mg/kg) as well as for the vehicle are also given.

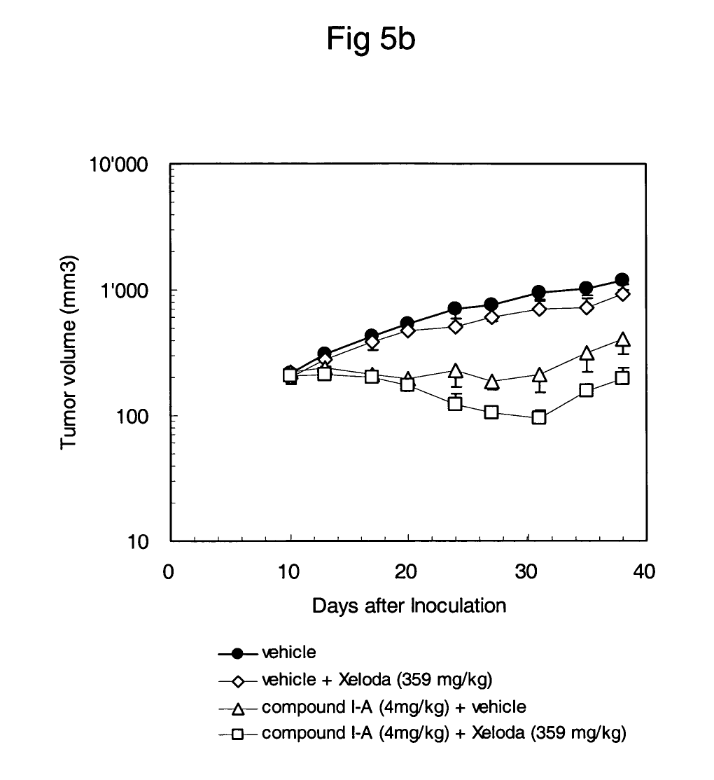

### DETAILED DESCRIPTION

Definitions

Herceptin™ (Trastuzumab) is a recombinant DNA-derived, humanized monoclonal antibody that selectively binds with high affinity to the extracellular domain of the human epidermal growth factor receptor 2 protein, HER2.

Omnitarg™ (Pertuzumab; 2C4) is a humanized antibody known as HER dimerization inhibitor (HDIs). HDIs block the ability of the HER2 receptor to collaborate with other HER receptor family members (HER1/EGFR, HER3, and HER4). In cancer cells, interfering with HER2's ability to collaborate with other HER family receptors blocks cell signaling and may ultimately lead to cancer cell growth inhibition and death of the cancer cell. HDIs, because of their unique mode of action, have the potential to work in a wide variety of tumors, including those that do not overexpress HER2.

Xeloda™ (capecitabine) is a fluoropyrimidine carbamate with antineoplastic activity.

Camptosar™ (Topotecin™; irinotecan hydrochloride; CPT-11) is a semisynthetic, water-soluble derivative of camptothecin, which is a cytotoxic alkaloid extracted from plants such as Camptotheca acuminata. Irinotecan and its active metabolite, SN-38, inhibit the action of Topoisomerase I, an enzyme that produces reversible single-strand breaks in DNA during DNA replication.

Platinol™(Randa™; cis-diamminedichloroplatinum; CDDP; cisplatin) is an inorganic Platinum (II) complex. It is widely prescribed for a variety of tumors (germ-cell, advanced bladder carcinoma, adrenal cortex carcinoma, breast cancer, head and neck carcinoma, lung carcinoma). It is administered intravenously for one to 5 days in a row, followed by a rest period of 2-3 weeks.

The term “pharmaceutically acceptable salt” as used herein refers to conventional acid- or base-addition salts that retain the biological effectiveness and properties of the parent compounds, for example the compounds of formula (I). According to the present invention, acid-addition salts are especially preferred and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid, trifluoro acetic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g., Ansel, H., et. al., Pharmaceutical Dosage Forms and Drug Delivery Systems, 6th ed., 1995.

The term “antineoplastic” means inhibiting or preventing the development, maturation or proliferation of malignant cells.

As used herein the term “concomitant” or “simultaneously” means administration of both components during the same 24 hour period, preferably within one or two hours of each other.

As used herein, the term “patient” means any mammal, preferably a human.

As used herein “sequential” (as in sequential administration or sequenced over time) means that one component is administered more than 24 hours after the other component, preferably within 2-15 days of the other component.

As used herein the term “separate administration” means that one component may be administered first and the second component is administered thereafter, with no limitation as to the duration of time. Accordingly the term “separate administration” encompasses both the time limits of “concomitant” (“simultaneously”) and “sequential” (sequential administration or sequenced over time) administration.

As used herein, “therapeutically effective” means an amount of drug, or combination or composition, which is effective for producing a desired therapeutic effect upon administration to a patient, for example, to stem the growth, or result in the shrinkage, of a cancerous tumor.

“Therapeutic index” is a well-recognized term of art and is an important parameter in the selection of anticancer agents for clinical trial. Therapeutic Index takes into consideration the efficacy, pharmacokinetics, metabolism and bioavailability of anticancer agents. See, e.g., J. Natl. Cancer Inst. 81(13):988-94 (Jul. 5, 1989)

The term “vehicle” as used herein means the solution wherein the compound of formula I-A, alone or in combination with the respective medicament, or the respective medicament alone is dissolved for subsequent application. Further specification of such solutions as well as their use and effects according to the present invention is illustrated by the examples herein, which must in no way be considered as limiting the scope of the present invention.

Description

It has now been found that a combination of at least one Dolastatin 10 derivative, in particular at least one of formula (I) as defined below, show more than additive effects when administered together with one of the known anti cancer drugs Xeloda™, Herceptin™, Omnitarg™, Camptosar™(Topotecin™ in Japan) or Platinol™ (Randa™ in Japan) or pharmaceutically acceptable salts thereof.

Therefore, the present invention is directed to a method for the treatment of cancer via administration of a pharmaceutical composition, comprising at least one compound of formula (I) or a pharmaceutically acceptable salt thereof 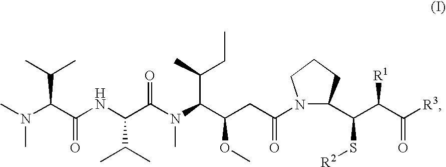
in combination with capecitabine; trastuzumab; pertuzumab; irinotecan or a pharmaceutically acceptable salt thereof; or cisplatin for simultaneous, sequential or separated administration in the treatment of cancer;
wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

In a preferred embodiment, the present invention is directed to a method for the treatment of cancer via administration of a pharmaceutical composition, comprising at least one compound of formula (I) or a pharmaceutically acceptable salt thereof 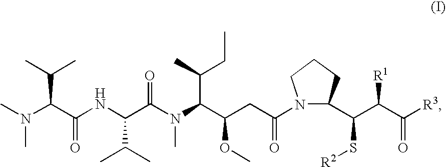
in combination with capecitabine, trastuzumab or pertuzumab for simultaneous, sequential or separate administration in the treatment of cancer;
wherein
R¹ and R² are methyl; ethyl; propyl; isopropyl or butyl;
R³ is phenylalkyl-, or phenyldialkylamino or phenylalkyloxy, having (C¹-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

The invention is also directed to a pharmaceutical composition, comprising at least one compound of formula (I) or a pharmaceutically acceptable salt thereof 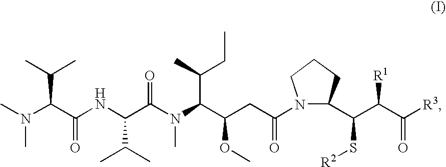
in combination with capecitabine; trastuzumab; pertuzumab; irinotecan or a pharmaceutically acceptable salt thereof; or cisplatin for simultaneous, sequential or separated administration in the treatment of cancer;
wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

In a preferred embodiment, the present invention is directed to a pharmaceutical composition, comprising at least one compound of formula (I) or a pharmaceutically acceptable salt thereof 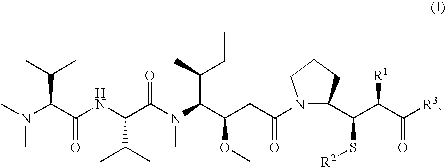
in combination with capecitabine, trastuzumab or pertuzumab for simultaneous, sequential or separated administration in the treatment of cancer;
wherein
R¹ and R² are methyl; ethyl; propyl; isopropyl or butyl;
R³ is phenylalkyl-, or phenyldialkylamino or phenylalkyloxy, having (C¹-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

With respect to the compounds of formula (I), the pharmaceutically acceptable salts which are formed with trifluoro acetic acid or hydrochloric acid are especially preferred.

A preferred embodiment of the present invention is the pharmaceutical composition as described above, comprising at least one compound of formula (I), wherein
R¹ and R² are methyl; and
R³ is as defined above.

Another preferred embodiment of the present invention is the pharmaceutical composition as defined above, wherein said compound of formula (I) is the compound of formula (I-A)

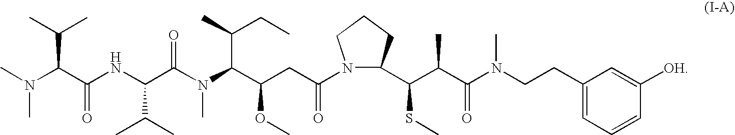

Another preferred embodiment of the present invention is the pharmaceutical composition as defined above, wherein said compound of formula (I) is administered in combination with trastuzumab.

Another preferred embodiment of the present invention is the pharmaceutical composition as defined above, wherein said compound of formula (I) is administered in combination with pertuzumab.

Another preferred embodiment of the present invention is the pharmaceutical composition as defined above, wherein said compound of formula (I) is administered in combination with capecitabine.

Another preferred embodiment of the present invention is the pharmaceutical composition as defined above, wherein said compound of formula (I) is administered in combination with cisplatin.

Another preferred embodiment of the present invention is the pharmaceutical composition as defined above, wherein said compound of formula (I) is administered in combination with irinotecan or a pharmaceutically acceptable salt thereof.

Another preferred embodiment of the present invention is the pharmaceutical composition as defined above, wherein said compound of formula (I) is administered in combination with irinotecan hydrochloride.

Another preferred embodiment of the present invention is the pharmaceutical composition as defined above, whereby said compound of formula (I) is administered simultaneously with capecitabine, trastuzumab, pertuzumab, cisplatin or irinotecan or a pharmaceutically acceptable salt thereof.

Another preferred embodiment of the present invention is the pharmaceutical composition as defined above, whereby said compound of formula (I) is administered simultaneously with capecitabine, trastuzumab or pertuzumab.

Another preferred embodiment of the present invention is the pharmaceutical composition as defined above, whereby said compound of formula (I) is administered simultaneously with cisplatin or irinotecan or a pharmaceutically acceptable salt thereof.

Another preferred embodiment of the present invention is the pharmaceutical composition as defined above, whereby said compound of formula (I) is administered simultaneously with irinotecan hydrochloride.

Another preferred embodiment of the present invention is the pharmaceutical composition as defined above, whereby said compound of formula (I) is administered sequentially with capecitabine, trastuzumab, pertuzumab, cisplatin or irinotecan or a pharmaceutically acceptable salt thereof.

Another preferred embodiment of the present invention is the pharmaceutical composition as defined above, whereby said compound of formula (I) is administered sequentially with capecitabine, trastuzumab or pertuzumab.

Another preferred embodiment of the present invention is the pharmaceutical composition as defined above, whereby said compound of formula (I) is administered sequentially with cisplatin or irinotecan or a pharmaceutically acceptable salt thereof.

Another preferred embodiment of the present invention is the pharmaceutical composition as defined above, whereby said compound of formula (I) is administered sequentially with irinotecan hydrochloride.

Another preferred embodiment of the present invention is the pharmaceutical composition as defined above, whereby said compound of formula (I) is administered separately from capecitabine, trastuzumab, pertuzumab, cisplatin or irinotecan or a pharmaceutically acceptable salt thereof.

Another preferred embodiment of the present invention is the pharmaceutical composition as defined above, whereby said compound of formula (I) is administered separately from capecitabine, trastuzumab or pertuzumab.

Another preferred embodiment of the present invention is the pharmaceutical composition as defined above, whereby said compound of formula (I) is administered separately from cisplatin or irinotecan or a pharmaceutically acceptable salt thereof.

Another preferred embodiment of the present invention is the pharmaceutical composition as defined above, whereby said compound of formula (I) is administered separately from irinotecan hydrochloride.

Another preferred embodiment of the present invention is the use of a pharmaceutical composition as described above for the treatment of cancer.

Another preferred embodiment of the present invention is the use of a pharmaceutical composition as described above for the treatment of solid tumors.

Another preferred embodiment of the present invention is the use of a pharmaceutical composition as described above for the treatment of colorectal cancer, prostate cancer, pancreatic cancer, breast cancer or lung cancer.

Another preferred embodiment of the present invention is the use of a pharmaceutical composition as described above for the treatment of colorectal cancer, prostate cancer, pancreatic cancer or breast cancer.

Another preferred embodiment of the present invention is the use of a pharmaceutical composition as described above for the treatment of lung cancer, preferably non-small cell lung cancer (NSCLC).

Another preferred embodiment of the present invention is the use of a pharmaceutical composition as described above, containing a compound of formula (I) or (I-A) together with cisplatin or irinotecan or a pharmaceutically acceptable salt thereof for the treatment of lung cancer, preferably non-small cell lung cancer (NSCLC).

Another preferred embodiment of the present invention is the use of a pharmaceutical composition as described above, containing a compound of formula (I) or (I-A) together with irinotecan hydrochloride for the treatment of lung cancer, preferably non-small cell lung cancer (NSCLC).

Another preferred embodiment of the present invention is the use of a pharmaceutical composition as described above for the production of a medicament for the treatment of cancer.

Another preferred embodiment of the present invention is the use of a pharmaceutical composition as described above for the production of a medicament for the treatment of solid tumors.

Another preferred embodiment of the present invention is the use of a pharmaceutical composition as described above for the production of a medicament for the treatment of colorectal cancer, prostate cancer, pancreatic cancer, breast cancer or lung cancer.

Another preferred embodiment of the present invention is the use of a pharmaceutical composition as described above for the production of a medicament for the treatment of colorectal cancer, prostate cancer, pancreatic cancer or breast cancer.

Another preferred embodiment of the present invention is the use of a pharmaceutical composition as described above for the production of a medicament for the treatment of lung cancer.

Another preferred embodiment of the present invention is the use of a pharmaceutical composition as described above for the production of a medicament for the treatment of non-small cell lung cancer.

In particular, the present invention relates to the treatment of cancer with therapeutic, more than additive combinations of N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) and capecitabine, trastuzumab, pertuzumab, cisplatin and irinotecan or a pharmaceutically acceptable salt thereof; and the use of such combinations for an improved treatment of cancer, especially solid tumors, more particularly those defined above.

More particularly, the present invention relates to the treatment of cancer with therapeutic, more than additive combinations of N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl) -2-methyl-propyl]-2-dimethylamino-3-methyl-butyramide (compound of formula (I-A)) and capecitabine, trastuzumab or pertuzumab; and the use of such combinations for an improved treatment of cancer, especially solid tumors, more particularly those defined above.

More particularly, the present invention relates to the treatment of cancer with therapeutic, more than additive combinations of N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl) -2-methyl-propyl]-2-dimethylamino-3-methyl-butyramide (compound of formula (I-A)) and cisplatin or irinotecan or a pharmaceutically acceptable salt thereof; and the use of such combinations for an improved treatment of cancer, especially solid tumors, more particularly those defined above.

More particularly, the present invention relates to the treatment of lung cancer with therapeutic, more than additive combinations of N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) and irinotecan hydrochloride; and the use of such combinations for an improved treatment of lung cancer, especially non-small cell lung cancer.

It was unexpectedly found that administration of each of capecitabine, trastuzumab, pertuzumab, cisplatin or irinotecan or a pharmaceutically acceptable salt thereof with N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide in accordance with the present invention results in improved, more than additive antineoplastic effects. This means that said antineoplastic effects are significantly superior to the results obtained by simple addition of the effects of each compound alone. Namely, administration of each combination in accordance with the present invention resulted in an improved therapeutic index (that is, superior efficacy) in comparison to either component alone without a significant increase in toxicity, as i.e. judged by the body weight control during the studies. Alternatively, the invention permits reduction of the amount of at least one component (in comparison the amount typically given in monotherapy) while retaining a desirable therapeutic index. In preferred embodiments, the amount of both components (in comparison to the amount typically given in monotherapy) is reduced affording reduced toxicity while still retaining a desirable therapeutic index.

The invention relates more particularly to a method of treating cancer, said method comprising administering to a patient in need thereof an effective amount of a therapeutic composition containing at least a first component consisting of the compound of formula (I) or (I-A), and a second component consisting of capecitabine, trastuzumab, pertuzumab, cisplatin or irinotecan or a pharmaceutically acceptable salt thereof; said method being characterized in that said composition shows a more than additive effect with respect to the effect of each of its active components alone.

Further to this, the invention relates to a method of treating cancer, said method comprising administering to a patient in need thereof an effective amount of a therapeutic composition containing at least a first component consisting of the compound of formula (I) or (I-A), and a second component consisting of capecitabine, trastuzumab or pertuzumab; said method being characterized in that said composition shows a more than additive effect with respect to the effect of each of its active components alone.

The invention relates more particularly to a method of treating non-small cell lung cancer, said method comprising administering to a patient in need thereof an effective amount of a therapeutic composition containing at least a first component consisting of the compound of formula (I) or (I-A), and a second component consisting of cisplatin or irinotecan or a pharmaceutically acceptable salt thereof; said method being characterized in that said composition shows a more than additive effect with respect to the effect of each of its active components alone.

According to the method of the present invention, the two components: the compound of formula (I) or (I-A) and capecitabine, trastuzumab, pertuzumab, cisplatin or irinotecan or a pharmaceutically acceptable salt thereof may be administered simultaneously, separately or sequenced over time. When administered separately, the compound of formula (I) or (I-A) may be administered first or capecitabine, trastuzumab, pertuzumab, cisplatin or irinotecan or a pharmaceutically acceptable salt thereof may be administered first.

Therapeutic compositions as defined above comprise effective therapeutic quantities of at least the compound of formula (I) or (I-A) and capecitabine, trastuzumab, pertuzumab, cisplatin or irinotecan or a pharmaceutically acceptable salt thereof and pharmaceutically acceptable supports to form together or separately liquid composition(s) such as solutions or suspension, as such or in a capsule; or solid composition(s) such as compressed tablets, pills, powders and the like. The preferred doses and dosage forms correspond to those typically recommended in monotherapy of each of the above-mentioned product, and are readily available to the skilled artisan from the corresponding publications. Preferably, the administration of the compound of formula (I) or (I-A) and the second additional component is carried out according to a regimen dependent on the type of cancer and more particularly on the type of tumors. The dosage ranges for the administration of the combination of the compound of formula (I) or (I-A) and capecitabine, trastuzumab, pertuzumab, cisplatin or irinotecan or a pharmaceutically acceptable salt thereof according to the present invention may also vary with the administration routes, as well as the state of the patient (age, extent of the disease).

Irrespective of the use of the administration, i.e. simultaneously, separately or sequenced over time (i.e., sequentially), the two components, the compound of formula (I) or (I-A) and capecitabine, trastuzumab, pertuzumab, cisplatin or irinotecan or a pharmaceutically acceptable salt thereof, can be administered by identical or different administration routes. They can be administered by identical or different administration routes when they are present in separated form, and by identical administration routes when they are present in unseparated form.

Capecitabine, trastuzumab, pertuzumab, cisplatin or irinotecan hydrochloride are known to the skilled artisan. Their preferred doses and dosage forms are well documented and readily available to the one skilled in the art. Preferred doses according to the present invention are 5 to 50 mg/kg, preferably 15 to 30 mg/kg trastuzumab; 5 to 60 mg/kg, preferably 20 to 40 mg/kg pertuzumab; and 50 to 550 mg/kg, preferably 150 to 400 mg/kg, more preferably from 300 to 400 mg/kg capecitabine. Cisplatin is used at doses between 0.5 and 10 mg/kg, preferably between 4 and 7 mg/kg; and irinotecan hydrochloride at a doses between 10 to 120 mg/kg, preferably 30 to 100 mg/kg, more preferably 60 to 90 mg/kg.

For the compound of formula (I-A) the maximum tolerated dose (MTD) was determined to be 6 mg/kg in the studies according to the present invention. Therefore the compound of formula (I-A) can be administered at doses between 0.1 and 6 mg/kg, preferably at doses between 1 and 6 mg/kg, more preferably at doses of 3, 4 and 6 mg/kg. It is understood that some compounds of formula (I) can show higher MTD's and therefore applicable doses of more than 6 mg/kg without departing from the spirit of the present invention.

In particular the following results were observed for each of the three combinations according to the present invention:

Compound of Formula (I-A) with Trastuzumab (Herceptin™; KPL-4 Xenograft Model):

Contrary to the results of monotherapy with either the compound of formula (I-A) or trastuzumab, the combination studies of 3, 4 and 6 mg/kg of the compound of formula (I-A) with 20 mg/kg trastuzumab resulted in significant tumor regression of longer duration, at least until day 70 of the treatment at all dosages, in the KPL-4 human breast carcinoma xenograft model. Palpable tumors were not present after day 28 at 3 mg/kg (½ MTD) or 4 mg/kg (⅔ MTD) of the compound of formula (I-A) plus 20 mg/kg (MTD) trastuzumab. Observation of the antitumor activity in mice with the combination of the compound of formula (I-A) and trastuzumab was continued until day 252. The combination of 6 mg/kg (MTD) of the compound of formula (I-A) plus 20 mg/kg (MTD) trastuzumab also resulted in potent tumor regression. Furthermore, complete tumor regression was observed in 2 out of 5 mice at 3 mg/kg and all 5 mice at 4 or 6 mg/kg of the compound of formula (I-A) plus 20 mg/kg trastuzumab.

To quantify the potency of each drug in combination, statistical analyses were performed with data collected at one week after the final injection. The volume of tumor treated with the compound of formula (I-A) at 3, 4, or 6 mg/kg decreased significantly in comparison with that of the 2.5% DMSO-saline plus 20 mg/kg human IgG control. In addition, tumor volumes in combination therapies of 3, 4, or 6 mg/kg of the compound of formula (I-A) with 20 mg/kg trastuzumab were compared to those of the monotherapy of the compound of formula (I-A) on day 21: every combination showed superior antitumor activity compared to that of monotherapy.

Based on these findings, a particularly preferred embodiment of the present invention is the combination of 3 mg/kg of the compound of formula (I-A) with 20 mg/kg trastuzumab.

Another particularly preferred embodiment of the present invention is the combination of 4 or 6 mg/kg of the compound of formula (I-A) with 20 mg/kg trastuzumab.

Yet another preferred embodiment of the present invention is the combination as defined above for the manufacture of medicaments for the treatment of breast cancer.

Compound of Formula (I-A) with Pertuzumab (Omnitarg™; KPL-4 Xenograft Model):

In these combination studies, using the KPL-4 human breast carcinoma xenograft model, significant tumor regression was observed at all dosages of the compound of formula (I-A). No evidence of tumors was detected by palpation after day 28 at 4 mg/kg of the compound of formula (I-A) plus 30 mg/kg pertuzumab. Observation of the antitumor activity in mice with the combination of the compound of formula (I-A) and pertuzumab was continued until day 133. The combination of 4 mg/kg of the compound of formula (I-A) plus 30 mg/kg pertuzumab showed potent tumor regression. Furthermore, the tumors in 3 out of 5 mice at 4 mg/kg of the compound of formula (I-A) plus 30 mg/kg pertuzumab completely regressed.

To assess the potency of each drug in combination, statistical analyses were performed using results on tumor volume assessed at one week after the final injection. The volume of tumor treated with the compound of formula (I-A) at 3, 4, or 6 mg/kg decreased strongly in comparison with that of the control; significant differences were observed on day 21 using the Tukey test (P=0.0038 at 3 mg/kg, and P<0.0001 for 4 and 6 mg/kg). In addition, tumor volumes in combination therapies of 3, 4, or 6 mg/kg of the compound of formula (I-A) with pertuzumab were compared to those of the monotherapy of the compound of formula (I-A) on day 21: every combination showed superior antitumor activity compared to that of the compound of formula (I-A) monotherapy.

Based on these findings, a particularly preferred embodiment of the present invention is the combination of 4 mg/kg of the compound of formula (I-A) with 30 mg/kg pertuzumab.

Yet another preferred embodiment of the present invention is the combination as defined above for the manufacture of medicaments for the treatment of breast cancer.

Compound of Formula (I-A) with Capecitabine (Xeloda™; MAXF-401 Xenograft Model):

The antitumor activity of each dose (3, 4, or 6 mg/kg) of the compound of formula (I-A) in combination with 359 mg/kg capecitabine showed to be equal to that of either of the single agents in the MAXF-401 human breast carcinoma xenograft model. The combination of each dose of the compound of formula (I-A) plus 359 mg/kg (MTD) capecitabine showed less favorable effects.

To evaluate the antitumor effects, statistical analyses were performed using results on tumor volume assessed at one week after the final injection. The volume of tumor treated with the compound of formula (I-A) at 3, 4, or 6 mg/kg decreased significantly (or more than that observed) in the 2.5% DMSO control. In addition, tumor volumes in combination therapies of 3, 4, or 6 mg/kg of the compound of formula (I-A) with capecitabine were compared to those of the monotherapy of the compound of formula (I-A) on day 21: combinations resulted insimilar antitumor activity to that observed with monotherapy. However, the duration of observed regression was longer with combination therapy than with capecitabine monotherapy.

Based on these findings, a particularly preferred embodiment of the present invention is the combination of 4 mg/kg of the compound of formula (I-A) with 359 mg/kg capecitabine.

Yet another preferred embodiment of the present invention is the combination as defined above for the manufacture of medicaments for the treatment of breast cancer.

Compound of Formula (I-A) with Cisplatin (Platinol™, CDDP; NCI-H460 Xenograft Model):

Antitumor activity the compound of formula (I-A) in combination with cisplatin (CDDP) in the NCI-H460 human large cell lung carcinoma xenograft model was examined. The compound of formula (I-A) was administered weekly for 3 consecutive weeks and CDDP was administered at the first day of the treatment. At the dose of 2 mg/kg (⅓ of maximum tolerated dose, MTD) and 3 mg/kg (½ MTD), the compound of formula (I-A) reduced the tumor growth by 45% and 93% respectively at day 12. At the dose of 5 mg/kg (½ MTD) and 6.7 mg/kg (⅔ MTD), CDDP reduced the tumor growth by 56% and 66% respectively at day 12. Combination of 2 mg/kg of the compound of formula (I-A) and 6.7 mg/kg CDDP reduced the tumor growth by 104% which was greater than that observed with the corresponding dose of the compound of formula (I-A) or CDDP alone without toxicity judged by the body weight loss at day 12. Combination of 3 mg/kg of the compound of formula (I-A) and 5 mg/kg CDDP reduced the tumor growth by 108% which was greater than the corresponding dose of the compound of formula (I-A) or CDDP alone without toxicity judged by the body weight loss at day 12.

Therefore, a preferred embodiment of the present invention is the combination of 2 mg/kg of the compound of formula (I-A) together with 6.7 mg/kg CDDP.

Yet another preferred embodiment of the present invention is the combination of 3 mg/kg of the compound of formula (I-A) and 5 mg/kg CDDP.

Still another preferred embodiment of the present invention is a combination as defined above for the manufacture of medicaments for the treatment of non-small cell lung cancer.

Compound of Formula (I-A) with Irinotecan (Camptosar™; Calu-6 Xenograft Model):

Antitumor activity of the compound of formula (I-A) in combination with irinotecan was determined using the Calu-6 human lung cancer xenograft model. According to previous experiments, the maximum tolerated dose (MTD) of compound (I-A) was defined as 6 mg/kg and of irinotecan as 120 mg/kg. The compound of formula (I-A) was administered weekly for 3 consecutive weeks and irinotecan was administered weekly for 2 consecutive weeks followed by rest for a week. Doses of 0.375 mg/kg, 0.75 mg/kg, 1.5 mg/kg, and 3mg/kg of the compound of formula (I-A) inhibited tumor growth, dependent on the dose by 11%, 21%, 41%, and 117% respectively, and measured at day 32 after the inoculation. At 80 mg/kg (⅔ MTD) of irinotecan, growth of the tumor was inhibited by 68% at day 32. Antitumor activity of 1.5 mg/kg (¼ MTD) of the compound of formula (I-A) in combination with 80 mg/kg (⅔ MTD) of irinotecan was 110% at day 32. This effect is more than additive with respect to the single agents. No toxicity, as judged by the body weight loss, was observed in this combination. Antitumor activity of 0.375 mg/kg ( 1/16 MTD) or 0.75 mg/kg (⅛ MTD) of the compound of formula (I-A) in combination with 80 mg/kg irinotecan was consistent with those of the irinotecan alone at day 32. The combination of the 3 mg/kg (½ MTD) of the compound of formula (I-A) and 80 mg/kg (⅔ MTD) irinotecan was toxic.

Therefore, a preferred embodiment of the present invention is the combination of 1.5 mg/kg of the compound of formula (I-A) together with 80 mg/kg irinotecan hydrochloride.

Yet another preferred embodiment of the present invention is the use of the combination as defined above for the manufacture of medicaments for the treatment of non-small lung cancer.

Compound of Formula (I-A) with Capecitabine (Xeloda™; HT-29 Xenograft Model):

Antitumor activity of the compound of formula (I-A) in combination with capecitabine in the HT-29 human colorectal adenocarcinoma xenograft model was examined. The compound of formula (I-A) was administered weekly for 3 consecutive weeks and capecitabine was administered daily for 14 days. At the dose of 3 mg/kg (½ of maximum tolerance dose, MTD) and 4 mg/kg (⅔ MTD), the compound of formula (I-A) reduced the tumor growth by 76% and 101% respectively at day 31. At the dose of 359 mg/kg (2/3 MTD) and 539 mg/kg (MTD), capecitabine reduced the tumor growth by 32% and 50% respectively at day 31. Combination of 3 mg/kg (½ MTD) of the compound of formula (I-A) and 539 mg/kg (MTD) capecitabine reduced the tumor growth by 111%. Combination of 4 mg/kg (⅔ MTD) of the compound of formula (I-A) and 359 mg/kg (⅔ MTD) capecitabine reduced the tumor growth by 115% at day31.

Increased antitumor activity was observed with these combinations than with the corresponding dose of the compound of formula (I-A) or capecitabine alone without toxicity judged by minimum body weight loss at day 31.

Therefore, a preferred embodiment of the present invention is the combination of 3 mg/kg of the compound of formula (I-A) together with 539 mg/kg capecitabine.

Another preferred embodiment of the present invention is the combination of 4 mg/kg of the compound of formula (I-A) together with 359 mg/kg capecitabine.

Yet another preferred embodiment of the present invention is the use of either of the two combinations as defined above for the manufacture of medicaments for the treatment of colorectal cancer.

Unless defined in another manner, all the technical and scientific terms and abbreviations used herein have the meanings as commonly understood by a person of ordinary skill in the field to which the invention belongs. Similarly, all publications, Patent Applications, all Patents and all other references mentioned here are incorporated by way of reference.

### EXAMPLES

The following examples are provided for illustratory purposes and are not intended to limit the scope of applicants' invention.

### Example 1

### N-[1-(1-sec-Butyl-4-[2-(2-1[2-(3-hydroxy-phenyl)-ethyl]-methyl-carbamoyll-1-methylsulfanyl-propyl)-pyrrolidin-1-yl]-2-methoxy-4-oxo-butyll-methyl-carbamoyl)-2-methyl-propyl]-2-dimethylamino-3-methyl-butyramide (compound of formula (I-A)) in combination with trastuzumab (Herceptin™)

Human Tumor Cell Line

The KPL-4 human breast carcinoma cell line was kindly donated by Prof. Kurebayashi, Kawasaki Medical Center. The cells were cultured in Dulbecco's MEM (Sigma Chemical Co.) supplemented with 5% (v/v) fetal bovine serum (Roche Diagnostics).

Animals

One hundred forty (140) 5-week-old female athymic nude mice (BALB/c nu/nu) were purchased from Charles River Japan, Inc. (Yokohama, Japan). The mice were housed in an air-conditioned room (temperature 22±2° C., relative humidity 55±10%) under 12 h light/dark cycles. The mice were given sterilized CE-2 and tap water with 25μg/mL gentamycin ad libitum. After 14 days quarantine in the animal facility of Nippon Roche Research Center (presently Kamakura Research Laboratories of Chugai Pharmaceutical Co., Ltd.), mice were subjected to the experiment.

Drugs

The title compound (compound of formula (I-A)) was synthesized at Kamakura Research Laboratories of Chugai Pharmaceutical Co., Ltd. Trastuzumab (Herceptin) was purchased from Chugai Pharmaceutical Co., Ltd. The title compound was dissolved in dimethyl sulfoxide (DMSO, Wako Pure Chemical Industries Ltd.) and diluted into 2.5% DMSO with saline (for injection, Ohtsuka Pharmaceutical Co., Ltd.) before administration. Trastuzumab was dissolved and diluted with saline before administration. Purified Human IgG (ICN Pharmaceuticals, Inc.) was dissolved and diluted into 20 mg/mL with saline for a stock solution. Before administration, the stock solution was diluted with saline for a dosage of 20 mg/kg.

Determination of Antitumor Activity

The in vivo evaluation procedure for anticancer drugs was based on the methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. A single cell suspension of KPL-4 (4×10⁶ cells per mouse), conventionally cultured in flasks, was implanted orthotopically at a volume of 100 μL under the nipple of the second mammary fat pad on the right of athymic nude mice. Mice bearing tumors of between 225 and 485 mm³ were selected on day 43 after tumor implantation and were randomly divided into 8 groups, consisting of 5 mice each. Mean tumor volume and body weight of the selected mice were 340 mm³ and 18.6 g respectively. Drug administration was initiated after grouping the mice. The title compound (3 mg/kg, 4 mg/kg, or 6 mg/kg) or its vehicle was administered (0.2 mL/mouse) intravenously once a week for 3 consecutive weeks. Trastuzumab or the IgG solution was administered (0.2 mL/mouse) intraperitoneally twice a week for 3 weeks on days 0, 4, 7, 11, 14, and 18. Mice in the control group were given the same volume of vehicle as those in the treatment groups: 0.2 mL of 2.5% DMSO in saline for the title compound and 0.2 mL of 20 mg/kg human IgG for trastuzumab. The tumor volume and body weight of each mouse were measured twice a week. Tumor volume was estimated by using the equation ab²/2, where a and b represent tumor length and width, respectively. One week after the final injection, tumor growth inhibition (TGI) was calculated using the formula (1-T/C)×100 (%), where T (treated group) and C (control group) represent the mean tumor volume change. ED₅₀ values were calculated from values of tumor growth inhibition on day 21 using the XL fit program. The maximum tolerated dose (MTD) was defined as the dose with neither lethality nor more than 20% body weight loss.

Statistical Analysis

Packaged software (SAS preclinical package version 5.0,SAS Institute Japan, Ltd.) was used for the statistical analysis. To evaluate the potency of the drugs, values of individual tumor volumes with treatments were compared to those of the controls using the Tukey test. ED₅₀s were calculated from values of tumor growth inhibition percentage using XL fit program #509 (MS Excel Add Version 3.0.5 Build-In: XLfit3 12, ID Business Solutions Ltd.)

### Example 2

### N-[1-({1-sec-Butyl-4-[2-(2-1[2-(3-hydroxy-phenyl)-ethyl]-methyl-carbamoyl}-1-methylsulfanyl-propyl)-pyrrolidin-1-yl]-2-methoxy-4-oxo-butyll-methyl-carbamoyl)-2-methyl-propyl]-2-dimethylamino-3-methyl-butyramide (compound of formula (I-A)) in combination with pertuzumab (Omnitarg™)

Human Tumor Cell Line

The KPL-4 human breast carcinoma cell line was kindly donated by Prof. Kurebayashi, Kawasaki Medical Center. The cells were cultured in Dulbecco's MEM (Sigma Chemical Co.) supplemented with 5% (v/v) fetal bovine serum (Roche Diagnostics).

Animals

One hundred (100) 5-week-old female athymic nude mice (BALB/c nu/nu) were purchased from Charles River Japan, Inc. (Yokohama, Japan). The mice were housed in an air-conditioned room (temperature 22±2° C., relative humidity 55±10%) under 12 h light/dark cycles. The mice were given sterilized CE-2 and tap water with 25μg/mL gentamycin ad libitum. After 14 days quarantine in the animal facility of Nippon Roche Research Center (presently Kamakura Research Laboratories of Chugai Pharmaceutical Co., Ltd.), mice were subjected to the experiment.

Drugs

The title compound (compound of formula (I-A)) was synthesized at Kamakura Research Laboratories of Chugai Pharmaceutical Co., Ltd. Pertuzumab was kindly given by Roche Diagnostics GmbH. The title compound was dissolved in dimethyl sulfoxide (DMSO, Wako Pure Chemical Industries Ltd.) and diluted into 2.5% DMSO with saline (for injection, Ohtsuka Pharmaceutical Co., Ltd.) before administration. Pertuzumab was dissolved and diluted with buffer solution before administration. Purified Human IgG (ICN Pharmaceuticals, Inc.) was dissolved and diluted into 25 mg/mL with saline for a stock solution. Before administration, the stock solution was diluted with saline for a dosage of 30 mg/kg.

Determination of Antitumor Activity

The In vivo evaluation procedure for anticancer drugs was based on methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. A single cell suspension of KPL-4 (4.5×10⁶ cells per mouse), conventionally cultured in flasks, was implanted orthotopically at a volume of 100 μL under the nipple of the second mammary fat pad on the right of athymic nude mice. Mice bearing tumors of between 190 and 356 mm³ were selected on day 18 after tumor implantation and were randomly divided into 9 groups, consisting of 5 mice each. Mean tumor volume and body weight of the selected mice were 226 mm³ and 20 g, respectively. Drug administration was initiated after grouping the mice. The title compound (3 mg/kg, 4 mg/kg, or 6 mg/kg) or its vehicle was administered (0.2mL/mouse) intravenously once a week for 3 consecutive weeks. Pertuzumab or the IgG solution was administered (0.2 mL/mouse) intraperitoneally once a week for 3 weeks on days 0, 7, and 14. Mice in the control group were given the same volume of vehicle as those in the treatment groups: 0.2 mL of 2.5% DMSO in saline for the title compound and 0.2 mL of 25 mg/kg human IgG for pertuzumab. The tumor volume and body weight of each mouse were measured twice a week. Tumor volume was estimated by using the equation ab²/2, where a and b represent tumor length and width, respectively. One week after the final injection, tumor growth inhibition (TGI) was calculated using the formula (1-T/C)×100 (%), where T (treated group) and C (control group) represent the mean tumor volume change. ED₅₀ values were calculated from values of tumor growth inhibition on day 21 using the XL fit program. The maximum tolerated dose (MTD) was defined as the dose with neither lethality nor more than 20% body weight loss.

Statistical Analysis

Packaged software (SAS preclinical package version 5.0, SAS Institute Japan, Ltd.) was used for the statistical analysis. To evaluate the potency of the drugs, values of individual tumor volumes with treatments were compared to those of the controls using the Tukey test. ED₅₀s were calculated from values of tumor growth inhibition percentage using XL fit program #509 (MS Excel Add-In: XLfit3 Version 3.0.5 Build 12, ID Business Solutions Ltd.)

### Example 3

### N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) in combination with capecitabine (Xeloda™)

Human Tumor Cell Line

The MAXF-401 human breast carcinoma cell line was obtained from Prof Fiebig (University of Freiburg, Germany). This cell line was maintained in mice; the tumor fragments were implanted subcutaneously and serially passaged in mice prior to use.

Animals

One hundred (100) 5-week-old female athymic nude mice (BALB/c nu/nu) were purchased from Charles River Japan, Inc. (Yokohama, Japan). The mice were housed in an air-conditioned room (temperature 22±2° C., relative humidity 55±10%) under 12 h light/dark cycles. The mice were given sterilized CE-2 and tap water with 25μg/mL gentamycin ad libitum. After 5 days quarantine in the animal facility of Nippon Roche Research Center (presently Kamakura Research Laboratories of Chugai Pharmaceutical Co., Ltd.), mice were subjected to the experiment.

Drugs

The title compound and capecitabine were synthesized at Kamakura Research Laboratories of Chugai Pharmaceutical Co., Ltd. The title compound was dissolved in dimethyl sulfoxide (DMSO, Wako Pure Chemical Industries Ltd.) and diluted into 2.5% DMSO with saline (for injection, Ohtsuka Pharmaceutical Co., Ltd.) before administration. Capecitabine was dissolved and diluted with 5% Gum Arabic-40 mM Citrate buffer (pH 6) before administration.

Determination of Antitumor Activity

The in vivo evaluation procedure for anticancer drugs was based on the methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. Ten to twenty mg (ca. 2×2×2 mm³) of tumor fragments of MAXF401 were implanted subcutaneously in the right flank of each mouse. Mice bearing tumors of between 245 and 588 mm³ were selected on day 35 after tumor implantation and were randomly divided into 11 groups, consisting of 5 mice each. Mean tumor volume and body weight of the selected mice were 401 mm³ and 22 g respectively. Drug administration was initiated after grouping the mice. The title compound (3 mg/kg, 4 mg/kg, or 6 mg/kg) or its vehicle was administered (0.2 mL/mouse) intravenously once a week for 3 consecutive weeks. Capecitabine or its vehicle was administered (0.5 mL/mouse) orally once a day for 14 consecutive days. Mice in the control group were given the same volume of vehicle as those in the treatment groups: 0.2 mL of 2.5% DMSO in saline for the title compound and 0.5 mL of 5% Gum Arabic-40 mM Citrate buffer (pH 6) for capecitabine. The tumor volume and body weight of each mouse were measured twice a week. Tumor volume was estimated by using the equation ab²/2, where a and b represent tumor length and width, respectively. One week after the final injection, tumor growth inhibition (TGI) was calculated using the formula (1-T/C)×100 (%), where T (treated group) and C (control group) represent the mean tumor volume change. ED₅₀ values were calculated from values of tumor growth inhibition on day 21 using the XL fit program. MTD was defined as the dose with neither lethality nor more than 20% body weight loss.

Statistical Analysis

Packaged software (SAS preclinical package version 5.0,SAS Institute Japan, Ltd.) was used for the statistical analysis. To evaluate the potency of the drugs, values of individual tumor volumes with treatments were compared to those of the controls using the Tukey test. ED₅₀s were calculated from values of tumor growth inhibition percentage using XL fit program #509 (MS Excel Add Version 3.0.5 Build -In: XLfit3 12, ID Business Solutions Ltd.)

### Example 4

### N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) in combination with cisplatin (Randa™, CDDP)

Human Tumor Cell Line

The NCI-H460 human large cell lung carcinoma cell line was purchased from American Type Culture Collection (MD, U.S.A.). The cells were cultured in RPMI-1640 medium (cat. # R8758, lot 12K2310, exp 01/03; lot 82K2497, exp 08/03; Sigma-Aldrich corporation, MO, U.S.A.) supplemented with 10% (v/v) fetal bovine serum (cat. # 047115, lot # 31300124; Roche Diagnostics KK, Tokyo, Japan).

Animals

Sixty of 5-week-old male athymic nude mice (BALB/c nu/nu) were purchased from Charles River Japan, Inc. (Yokohama, Japan). All mice were kept for 8 days in the animal facility of Kamakura Research Laboratories of Chugai Pharmaceutical Co., Ltd. before subjection to the experiment.

Drugs

The title compound was synthesized at Kamakura Research Laboratories of Chugai Pharmaceutical Co., Ltd. CDDP (Randa™, lot # 924150) was obtained from Nippon Kayaku Co., Ltd. (Tokyo, Japan). The title compound was dissolved in dimethyl sulfoxide (DMSO) and diluted into 2.5 % DMSO solution with saline (for Injection; Ohtsuka Pharmaceutical Industry Co., Ltd., Tokushima, Japan) at the date of administration. CDDP was diluted with saline at the date of administration.

Determination of Antitumor Activity

The in vivo evaluation procedure for anticancer drugs was based on the methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. A single cell suspension of NCI-H460 (5.6×106 cells per mouse) was inoculated subcutaneously into the right flank of each mouse. The tumor volume was estimated by using the equation ab²/2, where a and b represent tumor length and width, respectively. Mice bearing tumors of between 150 and 245 mm³ were selected on day 7 after tumor inoculation and were randomly divided into 8 groups, consisting of 5 mice each. Mean tumor volume and body weight of the selected mice were 187 mm3 and 24.3 g, respectively. Drug administration was initiated after mice were divided into groups. The title compound (2 mg/kg or 3 mg/kg), CDDP (5 mg/kg or 6.7 mg/kg) and each vehicle were administered intravenously. Tumor volume and body weight of each mouse were measured twice a week. That dose was defined as toxic when at least one dead mouse was observed during the administration period or when half or more mice showed more than 20% continuous body weight loss compared to the initial treatment day during the administration period. Tumor growth inhibition was calculated using the equation (1-ΔT/ΔC)×100, where ΔT represents the difference in tumor volume from the initial treatment date of the treated group and ΔC represents the difference in tumor volume from the initial treatment date of the vehicle group.

### Example 5

### N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) in combination with irinotecan (Topotecin™)

Human Tumor Cell Line

The Calu-6 human lung carcinoma cell line was purchased from American Type Culture Collection (MD, U.S.A.). The cells were cultured in Eagle's MEM (Sigma-Aldrich corporation, MO, U.S.A.) supplemented with 0.1 milli mol/L Non-essential amino acids (Invitrogen corporation, CA, U.S.A.), 1 milli mol/L sodium pyruvate (Invitrogen corporation, CA, U.S.A.), and 10% (v/v) fetal bovine serum (Roche Diagnostics KK, Tokyo, Japan).

Animals

60 of 5-week-old male athymic nude mice (BALB/c nu/nu) were purchased from Charles River Japan, Inc. (Yokohama, Japan). All mice were kept for 8 days in the animal facility of Kamakura Research Laboratories of Chugai Pharmaceutical Co., Ltd. before subjection to the experiment.

Drugs

The title compound was synthesized at Kamakura Research Laboratories of Chugai Pharmaceutical Co., Ltd. Irinotecan (Topotecin™, lot. No. EQAZF13) was obtained from Daiichi Pharmaceutical Co., Ltd. (Tokyo, Japan). The title compound was dissolved in dimethyl sulfoxide (DMSO) and diluted into 2.5 % DMSO solution with saline (For Injection; Ohtsuka Pharmaceutical Industry Co., Ltd., Tokushima, Japan) at the date of administration. Irinotecan was diluted with saline at the date of administration.

Determination of Antitumor Activity

The in vivo evaluation procedure for anticancer drugs was based on the methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. A single cell suspension of Calu-6 (5.7×106 cells per mouse) was inoculated subcutaneously into the right flank of each mouse. The tumor volume was estimated by using the equation ab²/2, where a and b represent tumor length and width, respectively. Mice bearing tumors of between 181 and 326 mm³ were selected on day 11 after tumor inoculation and were randomly divided into 10 groups, consisting of 5 mice each. Mean tumor volume and body weight of the selected mice were 251 mm³ and 24.7 g respectively. Drug administration was initiated after mice were randomized and divided into groups. The tide compound (0.375 mg/kg; 0.75 mg/kg; 1.5 mg/kg or 3 mg/kg), irinotecan (80 mg/kg) and each vehicles were administered intravenously. Tumor volume and body weight of each mouse were measured twice a week. That dose was defined as toxic when at least one dead mouse was observed during the administration period or when half or more mice showed more than 20% continuous body weight loss compared to the initial treatment day during the administration period. Tumor growth inhibition was calculated by using the equation (1-ΔT/ΔC)×100, where ΔT represents the tumor volume difference from the initial treatment date of treated group and ΔC represents the tumor volume difference from the initial treatment date of vehicle group.

### Example 6

### N-[1-({1-sec-Butyl-4-[2-(2-{[2-(3-hydroxy-phenyl)-ethyl]-methyl-carbamoyl}-1-methylsulfanyl-propyl)-pyrrolidin-1-yl]-2-methoxy-4-oxo-butyl}-methyl-carbamoyl)-2-methyl-propyl]-2-dimethylamino-3-methyl-butyramide (compound of formula (I-A)) in combination with capecitabine (Xeloda™)

Human Tumor Cell Line

The HT-29 human colorectal adenocarcinoma cell line was purchased from American Type Culture Collection (Rockville, MD, U.S.A.). The cells were cultured in McCoy's 5a medium (Sigma-Aldrich Corporation, MO, U.S.A.) supplemented with 10% (v/v) fetal bovine serum (Roche Diagnostics KK, Tokyo, Japan).

Animals

Sixty of 5-week-old male athymic nude mice (BALB/c nu/nu) were purchased from Charles River Japan, Inc. (Yokohama, Japan). All mice were kept for 9 days in the animal facility of Kamakura Research Laboratories of Chugai Pharmaceutical Co., Ltd. before subjection to the experiment.

Drugs

The title compound (compound of formula I-A) was synthesized at Kamakura Research Laboratories of Chugai Pharmaceutical Co., Ltd. Capecitabine (Lot. No. 26954-190A-MIL) was purchased from F. Hoffinann-La Roche Ltd (Basel, Switzerland). The compound of formula I-A was dissolved in dimethyl sulfoxide (DMSO; Wako Pure Chemical Industries, Ltd., Osaka, Japan) and diluted into 2.5% DMSO solution with saline (for Injection; Ohtsuka Pharmaceutical Industry Co., Ltd., Tokushima, Japan) on the date of administration. Capecitabine was dissolved by the 40 milli mol/L citrate buffered-5% Gum arabic solution (pH 6.0; Chugai Pharmaceutical Co., Ltd.).

Determination of Antitumor Activity

The in vivo evaluation procedure for anticancer drugs was based on the methods described by Plowman, et al. (1997) for xenograft models. See, “Anticancer Drug Development Guide: Preclinical Screening, Clinical Trials, and Approval, Teicher B (ed) pp. 101-125, specifically Chap. 6, “Human Tumor Xenograft Models in NCI Drug Development”, hereby incorporated by reference. A single cell suspension of HT-29 (1.1×10⁷ cells per mouse) was inoculated subcutaneously into the right flank of each mouse. The tumor volume was estimated by using the equation ab²/2, where a and b represent tumor length and width, respectively. Mice bearing tumors of between 172 and 282 mm³ were selected on day 10 after tumor inoculation and were randomly divided into 7 groups, consisting of 6 mice each. Mean tumor volume and body weight of the selected mice were 217 mm³ and 25.0 g respectively. Drug administration was initiated after mice were divided into groups. The compound of formula I-A (3 mg/kg or 4 mg/kg) and its vehicle were administered intravenously. Capecitabine (359 mg/kg or 539 mg/kg) and its vehicle were administered orally. Tumor volume and body weight of each mouse were measured twice a week. The dose was defined as toxic when at least one dead mouse was observed during the administration period, or when half or more mice showed more than 20% continuous body weight loss compared to the initial treatment day during the administration period. Tumor growth inhibition was calculated using the equation (1-ΔT/ΔC)×100, where ΔT represents the difference in tumor volume from the initial treatment date of the treated group and ΔC represents the difference in tumor volume from the initial treatment date of the vehicle group.

1. A method of treating a patient suffering from cancer, comprising administering to the patient a therapeutically effective combination of a pharmaceutical composition comprising at least one compound of formula (I) 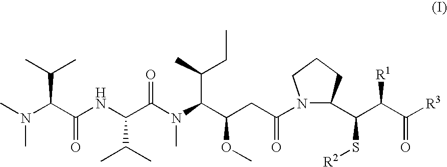
or a pharmaceutically acceptable salt of said compound and a known anti-cancer drug compound or pharmaceutically acceptable salt of said known anti-cancer drug compound,
wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

2. The method of claim 1, wherein the known anti-cancer drug compound is selected from the group consisting of capecitabine, trastuzumab, pertuzumab, irinotecan or cisplatin or a pharmaceutically acceptable salt thereof.

3. The method of claim 2, wherein the administration of the therapeutically effective combination is simultaneous, sequential or separate.

4. The method of claim 3, wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

5. The method of claim 4, wherein
R¹ and R² are methyl; and
R³ is as defined above.

6. The method of claim 5, wherein the said compound of formula (1) is the compound of formula (1A)

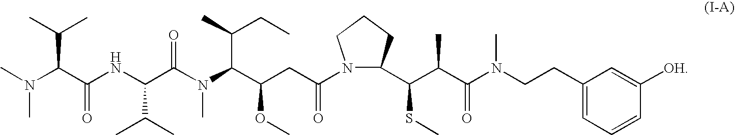

7. A method of treating cancer, said method comprising administering to a patient a therapeutically effective amount of a composition containing at least a first component consisting of the compound of formula I or a pharmaceutical salt thereof and a second component, wherein the second component is selected from the group consisting of capecitabine, trastuzumab, pertuzumab, cisplatin or irinotecan or a pharmaceutical salt thereof, said method being characterized in that said composition shows a more than additive effect with respect to the effect of the first and second components alone.

8. The method of claim 7 wherein the first and second components are administered simultaneously, separately or sequenced over time (sequentially).

9. The method of claim 8 wherein the second component is selected from the group consisting of capecitabine, trastuzumab or pertuzumab.

10. The method of claim 8 wherein the cancer is non-small lung cancer and the second component is selected from the group consisting of cisplatin or irinotecan or a pharmaceutical salt thereof.

11. A method of treating cancer, said method comprising administering to a patient a therapeutically effective amount of a composition containing at least a first component consisting of the compound of formula I-A or a pharmaceutical salt thereof and a second component, wherein the second component is selected from the group consisting of capecitabine, trastuzumab, pertuzumab, cisplatin or irinotecan or a pharmaceutical salt thereof, said method being characterized in that said composition shows a more than additive effect with respect to the effect of the first and second components alone.

12. The method of claim 11 wherein the first and second components are administered simultaneously, separately or sequenced over time (sequentially).

13. The method of claim 12 wherein the second component is selected from the group consisting of capecitabine, trastuzumab or pertuzumab.

14. The method of claim 12 wherein the cancer is non-small lung cancer and the second component is selected from the group consisting of cisplatin or irinotecan or a pharmaceutical salt thereof.

15. The method of claim 12 wherein the first component of the compound of formula I-A is administered in a dosage amount of about 0.1 mg/kg to about 6 mg/kg.

16. The method of claim 15 wherein the dosage amount of the compound of formula I-A is from about 1 mg/kg to about 6mg/kg.

17. The method of claim 12 wherein the second component is trastuzumab and wherein the trastuzumab is administered in a dosage amount of about 5 mg/kg to about 50 mg/kg.

18. The method of claim 17 wherein the trastuzumab is administered in a dosage amount of about 15 mg/kg to about 30 mg/kg.

19. The method of claim 12 wherein the second component is pertuzumab and wherein the pertuzumab is administered in a dosage amount of about 5 mg/kg to about 60 mg/kg.

20. The method of claim 19 wherein the pertuzumab is administered in a dosage amount of about 20 mg/kg to about 40 mg/kg.

21. The method of claim 12 wherein the second component is capecitabine and wherein the capecitabine is administered in a dosage amount of about 50 mg/kg to about 550 mg/kg.

22. The method of claim 21 wherein the capecitabine is administered in a dosage amount of about 150 mg/kg to about 400 mg/kg.

23. The method of claim 22 wherein the capecitabine is administered in a dosage amount of about 300 mg/kg to about 400 mg/kg.

24. The method of claim 12 wherein the second component is cisplatin and wherein the cisplatin is administered in a dosage amount of about 0.5 mg/kg to about 10 mg/kg.

25. The method of claim 24 wherein the cisplatin is administered in a dosage amount of about 4 mg/kg to about 7 mg/kg.

26. The method of claim 12 wherein the second component is irinotecan hydrocholoride and wherein the irinotecan hydrocholoride is administered in a dosage amount of about 10 mg/kg to about 120 mg/kg.

27. The method of claim 26 wherein the irinotecan hydrocholoride is administered in a dosage amount of about 30 mg/kg to about 100 mg/kg.

28. The method of claim 27 wherein the irinotecan hydrocholoride is administered in a dosage amount of about 60 mg/kg to about 90 mg/kg.

29. The method of claim 12 wherein the first component is the compound of formula I-A in the amount of about 3 mg/kg to about 6 mg/kg and the second component is trastuzumab in the amount of about 20 mg/kg.

30. The method of claim 12 wherein the first component is the compound of formula I-A in the amount of about 3 mg/kg to about 6 mg/kg and the second component is pertuzumab in the amount of about 30 mg/kg.

31. The method of claim 12 wherein the first component is the compound of formula I-A in the amount of about 3 mg/kg to about 6 mg/kg and the second component is capecitabine in the amount of about 359 mg/kg.

32. The method of claim 12 wherein the first component is the compound of formula I-A in the amount of about 2 mg/kg to about 3 mg/kg and the second component is cisplatin in the amount of about 5 mg/kg to about 6.7 mg/kg.

33. The method of claim 12 wherein the first component is the compound of formula I-A in the amount of about 0.75 mg/kg and the second component is irinotecan hydrocholoride in the amount of about 80 mg/kg.

34. The method of claim 33 wherein the first component is administered weekly for about 3 consecutive weeks and the second component is administered weekly for about 2 consecutive weeks followed by a week of rest.

35. The method of claim 12 wherein the first component is the compound of formula I-A in the amount of about 3 mg/kg to about 4 mg/kg and the second component is capecitabine in the amount of about 359 mg/kg to about 539 mg/kg and wherein further said first component is administered weekly for about 3 consecutive weeks and said second component is administered daily for about 14 days.

36. A pharmaceutical composition, comprising at least one compound of formula (I) or a pharmaceutically acceptable salt of a compound of 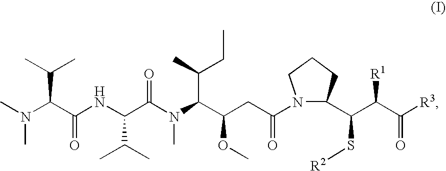
in combination with (one or at least one of the following) a known anti-cancer drug compound or a pharmaceutically acceptable salt of the known anti-cancer drug compound, for administration in the treatment of cancer,
wherein
R¹ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

37. The composition of claim 36, wherein the known anti-cancer drug compound is selected from the group consisting of capecitabine, trastuzumab, pertuzumab, irinotecan or cisplatin or a pharmaceutically acceptable salt thereof.

38. The composition of claim 37, wherein the administration is simultaneous, sequential or separated.

39. The composition of claim 38 wherein the known anti-cancer drug compound is selected from the group consisting of capecitabine, trastuzumab, pertuzumab and wherein
R₁ and R² are methyl; ethyl; propyl; isopropyl or butyl;
R³ is phenylalkyl-, or phenyldialkylamino or phenylalkyloxy, having (C₁-C₄)-alkylene and wherein the phenyl group optionally may be substituted with one, two or three substituents selected from the group consisting of halogen; alkoxycarbonyl; sulfamoyl; alkylcarbonyloxy; carbamoyloxy; cyano; mono- or di-alkylamino; alkyl; alkoxy; phenyl; phenoxy; trifluoromethyl; trifluoromethoxy; alkylthio; hydroxy; alkylcarbonylamino; 1,3-dioxolyl; 1,4-dioxolyl; amino and benzyl.

40. The composition of claim 38, wherein
R¹ and R² are methyl; and
R³ is as defined above.

41. The composition of claim 38, wherein the said compound of formula (1) is the compound of formula (1A)

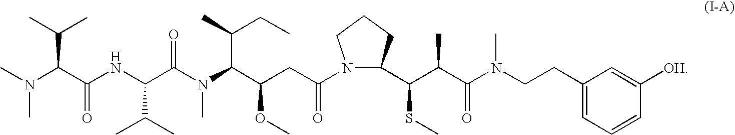

42. The pharmaceutical composition according to claim 38, wherein said compound of formula (I) is administered in combination with trastuzumab.

43. The pharmaceutical composition according to claim 38, wherein said compound of formula (I) is administered in combination with pertuzumab.

44. The pharmaceutical composition according to claim 38, wherein said compound of formula (I) is administered in combination with capecitabine.

45. The pharmaceutical composition according to claim 38, wherein said compound of formula (I) is administered in combination with cisplatin.

46. The pharmaceutical composition according to claim 38, wherein said compound of formula (I) is administered in combination with irinotecan or a pharmaceutically acceptable salt thereof.

47. The pharmaceutical composition according to claim 38, whereby said compound of formula (I) is administered simultaneously with capecitabine, trastuzumab, pertuzumab, cisplatin or irinotecan or a pharmaceutically acceptable salt thereof.

48. The pharmaceutical composition according to claim 38, whereby said compound of formula (I) is administered simultaneously with capecitabine, trastuzumab or pertuzumab.

49. The pharmaceutical composition according to claim 38, whereby said compound of formula (I) is administered simultaneously with cisplatin or irinotecan or a pharmaceutically acceptable salt thereof.

50. The pharmaceutical composition according to claim 38, whereby said compound of formula (I) is administered sequentially with capecitabine, trastuzumab, pertuzumab, cisplatin or irinotecan or a pharmaceutically acceptable salt thereof.

51. The pharmaceutical composition according to claim 38, whereby said compound of formula (I) is administered sequentially with capecitabine, trastuzumab or pertuzumab.

52. The pharmaceutical composition according to claim 38, whereby said compound of formula (I) is administered separately from capecitabine, trastuzumab, pertuzumab, cisplatin or irinotecan or a pharmaceutically acceptable salt thereof.

53. The pharmaceutical composition according to claim 38, whereby said compound of formula (I) is administered separately from capecitabine, trastuzumab or pertuzumab.

54. A method for the treatment of cancer comprising the administration of the pharmaceutical composition of claim 38.

55. The method of claim 54, wherein the cancer is selected from the group consisting of colorectal cancer, prostate cancer, pancreatic cancer, breast cancer or lung cancer.
